(19) United States
(12) Patent Application Publication
(10) Pub. No.: US 2023/0183446 A1
(43) Pub. Date: Jun. 15, 2023

(54) PROCESS FOR THE PRODUCTION OF HYDROPHOBIC AND REACTIVE INORGANIC AND/OR ORGANIC FILLERS, FILLERS PRODUCED IN THIS WAY AND MOLDINGS PRODUCED FROM A POLYMER-BASED CASTING COMPOUND CONTAINING AT LEAST ONE SUCH FILLER

(71) Applicant: Schock GmbH, Regen (DE)

(72) Inventors: Vitaliy DATSYUK, Zwiesel (DE); Adam ORENDORZ, Zwiesel (DE)

(21) Appl. No.: 18/071,864

(22) Filed: Nov. 30, 2022

(30) Foreign Application Priority Data
Dec. 9, 2021 (DE) 10 2021 132 486.6

(51) Int. Cl. C08K 3/013; C08K 5/00

(52) U.S. Cl. CPC C08K 3/013; C08K 5/0008; C08K 2201/006

(57) ABSTRACT

A process for producing hydrophobic and reactive, inorganic and/or organic fillers, including the steps of: (a) providing a filler having a defined surface area, (b) mixing the filler with the solution of at least one hydrophobizing and activating, biologically based reactive compound in a mixing assembly in an amount of 0.15×10-2 to 5.0×10-2 g per m2 filler surface area at a speed of 20 rpm to 200 rpm for 12 minutes to 120 minutes, (c) evacuating the hydrophobized and activated, inorganic and/or organic filler in a storage bag, a box or a drum, or directly in the casting compound.

### CROSS-REFERENCE TO RELATED APPLICATIONS

The present application claims priority of DE 10 2021 132 486.6, filed Dec. 9, 2021, the priority of this application is hereby claimed, and this application is incorporated herein by reference.

### BACKGROUND OF THE INVENTION

The invention relates to a process for producing hydrophobized and reactive, inorganic and/or organic fillers. Such fillers are used for example as adjuvants to polymer-based casting compounds from which composite moldings are produced.

Shrinkage and shrinkage stress are a major problem in numerous applications based on radically cured thermoset materials, such as, for example, molded kitchen sinks, wash basins, bath tubs or composite dental filling systems. Composite materials of these kinds generally include a radically cured polymer binder, an initiator system, and silane-treated inorganic filler particles. In the curing of these composite systems, shrinkage is observed which may give rise on the one hand to microcracks and on the other hand to severe intrinsic stresses in the material. In the case of molded kitchen sinks, for example, this may lead to crack propagation in the material, with the consequence of water leakage or of a reduction in the mechanical properties. The same problems may occur in dental composite materials: stresses, microleaks, detachment of adhesive, and, ultimately, pain for the patient.

This problem can be attributed to the high filler content of composite materials and to the use of 3-methacryloyloxypropyltrimethoxysilane, which is immobilized on the surface of the quartz particles and, with its relatively short triple chain, exhibits a relatively low mobility. During polymerization, this limited mobility leads to rapid breaking of the radicals and to the formation of short polymer chains, which because of the high stiffness leads to severe loading of the system. Thermally induced contractions or expansions of the material and also mechanical impacts may act as a source of microcracks at the filler-matrix interface.

The filler-matrix interface is where the natural stresses are concentrated, owing to the presence of numerous reactive groups at a rigid inorganic surface. The surface of the quartz sand particles generally has 0.8 hydroxyl groups per square nanometer of surface area. During the silanization process, almost every hydroxyl group reacts with an individual silane molecule and forms a unitary hydrophobic silane layer, leading to a superhydrophobic effect with very dense immobilization of the reactive methacrylate group. The high concentration of the double bonds immobilized on the filler surface results in the polymerization of the short polymer chains and hence in the formation of regions having a high inherent stress. The stresses can be reduced by replacing the methacryloylsilane with nonreactive silanes, but this reduces the mechanical properties such as the impact strength, for example, owing to the lack of bonding between filler and matrix.

The use of the silane-treated, inorganic and/or organic fillers which had been treated with a silane coupling agent necessitates the hydrolysis of the hydrolyzable ester functions of the silane. The hydrolysis of, for example, 1000 kg of 3-methacryloyl-propyltrimethoxysilane leads to the production of 387 kg of methanol, a flammable liquid with high vapor pressure, which can be fatal if swallowed.

Industrially, moreover, fillers are silanized by means of thermal activation at temperatures of 60° C. or higher. The energy needed for this process is generally realized via the combustion of natural gas or corresponding hydrocarbons, which contribute to emission of CO₂ and raise the process costs. After the silanization process, an aging time of several days is generally required until the desired hydrophobicity of the filler is established.

### SUMMARY AND DESCRIPTION OF THE INVENTION

It is an object of the present invention to eliminate the above-described technical and environmentally relevant problems of the prior art.

In order to achieve the object, a process is proposed for producing hydrophobic and reactive, inorganic and/or organic fillers, comprising the steps of: (a) providing a filler having a defined surface area, (b) mixing the filler with the solution of at least one hydrophobizing and activating, biologically based reactive compound in a mixing assembly in an amount of 0.15×10⁻² to 5.0×10⁻² g per m² filler surface area at a speed of 20 rpm to 200 rpm for 12 minutes to 120 minutes, (c) transferring the hydrophobized and activated, inorganic and/or organic filler into a storage bag, a box or a drum, or directly into the casting compound.

In contrast to the silanization process, which requires seven-day storage of the treated filler for the post-reaction, the present invention proposes a technology which allows the filler to be used directly after the treatment. Moreover, the process of the invention does not necessitate any heating process, whereas the silanization reaction is carried out with heating to at least 60° C. for at least 30 minutes.

Hydrophobized and activated fillers of the present invention have a different interface with the matrix. The double bond of the methacryloyl group, located close to the filler surface, encapsulates the filler surface during polymerization and keeps the double bond ready on the side chains of the fatty acid for copolymerization with the matrix. These chains lead to the development of the less-stressed filler-matrix interface. This reduces stresses locally and in the moldings overall.

The amounts of hydrophobizing, natural-based reactive substance to be activated are dependent on the specific filler surface area and are therefore indicated in g/m2. In this case the amount of methacryloyl monomer used is selected such that construction of a monolayer of the methacryloyl monomer via the amine group with the hydroxyl group of the filler is preferred. The amount is therefore dependent on the density of the hydroxyl groups on the filler surface. It is possible, for example, to assume 0.8-OH groups per nm² and 0.2 m²/g for a quartz sand, and to determine the required amount of methacryloyl monomer on that basis.

The mixing time in the mixing assembly, a drum hoop mixer for example, is likewise varied depending on the composition of the fillers. Particles having a larger diameter require less time in order to achieve uniform distribution of the oil-based monomers over the filler surface. In the case of fine particles, quartz or fruit stone flour, the mixing time is longer.

Provided in accordance with the invention is an inorganic and/or organic filler which has a hydrophobized and activated surface. The inorganic fillers may be selected from SiO₂, Al₂O₃, TiO₂, ZrO₂, Fe₂O₃, ZnO, Cr₂O₅, carbon, metals and metal alloys SiC, SiN, BN or mixtures thereof.

The inorganic fillers are ground fruit stones and/or fruit shells and may be selected from olive stones, peach stones, apricot stones, cherry stones, almond shells, argan shells, walnut shells, or a mixture thereof.

The inorganic and organic fillers may be used in a combination of both kinds of fillers. The mixing ratio may be selected as desired.

The hydrophobized and activated surface of the inorganic and/or organic filler is formed by immobilization of at least one biobased (meth)acrylated monomer, which comprises a fatty acid group originating from biological cultivation and esterified with the (meth)acryloyl group on the surface of the inorganic and/or organic filler.

The inorganic and organic fillers used in step (a) may have a particle size of 1 μm to 2000 μm. The present invention is based on the approach of hydrophobizing and activating inorganic and/or organic fillers, including a surface treatment with at least one biologically based reactive compound.

The hydrophobized and activated surface of the inorganic and/or organic filler is formed by immobilization of at least one biobased (meth)acrylated monomer, which contains a fatty acid group originating from biological cultivation and comprises a group esterified with the (meth)acryloyl group. The biobased (meth)acrylated monomer is dissolved in the monomer contained in the polymer matrix of the molding. The concentration of the biobased (meth)acrylated monomer ought to be 1 to 20 wt %, preferably 3 to 17.5 wt %, more particularly 5 to 15 wt %.

The hydrophobizing, biologically based reactive compound for activation, used in step (b), may be selected from the methacryloyl monomer based on oils of natural origin, of the general formula: H2C═C(R1)C(O)—NH—CH₂—CH₂—C(O)—O—R2, where R1 in the case of acryloyl is H and in the case of methacryloyl is CH3, where R2 is a fatty acid radical from the oils of natural origin, which reacts in the bulk with the N-hydroxyethyl-(meth)acrylamide.

Additionally the hydrophobizing, biobased reactive compound for activation that is used in step (b) may be dissolved in the at least one monomer present in the polymer matrix of the molding.

Solvents used may be monofunctional monomers in the form of an acrylate monomer. These may be selected from methyl acrylate, ethyl acrylate, n-butyl acrylate, tert-butyl acrylate, isobutyl acrylate, isodecyl acrylate, dihydroxycyclopentadienyl acrylate, ethyl diglycol acrylate, heptadecyl acrylate, 4-hydroxybutyl acrylate, 2-hydroxyethyl acrylate, hydroxyethylcaprolactone acrylate, polycaprolactone acrylate, hydroxypropyl acrylate, lauryl acrylate, stearyl acrylate, 2-(2-ethoxy)ethyl acrylate, tetrahydroxyfurfuryl acrylate, 2-phenoxyethyl acrylate, ethoxylated 4-phenyl acrylate, trim ethylcyclohexyl acrylate, octyldecyl acrylate, tridecyl acrylate, ethoxylate 4-nonylphenyl acrylate, isobornyl acrylate, cyclic trimethylolpropane formal acrylate, ethoxylated 4-lauryl acrylate, polyester acrylate, hyperbranched polyester acrylate, melamine acrylate, silicone acrylate, epoxy acrylate.

It is possible, furthermore, to use a monofunctional monomer in the form of a methacrylate. This may be selected from methyl methacrylate, ethyl methacrylate, n-butyl methacrylate, isobutyl methacrylate, tert-butyl methacrylate, behenyl methacrylate, behenylpolyethylene glycol methacrylate, cyclohexyl methacrylate, isodecyl methacrylate, 2-ethylhexyl methacrylate, lauryl methacrylate, stearyl methacrylate, stearylpolyethylene glycol methacrylate, isotridecyl methacrylate, ureidomethacrylate, tetrahydrofurfuryl methacrylate, phenoxyethyl methacrylate, isobornyl methacrylate, methoxypolyethylene glycol methacrylate, glycidyl methacrylate, glycerol formal methacrylate, lauryltetradecyl methacrylate.

As solvent it is also possible to use a polyfunctional monomer in the form of a polyfunctional acrylate. This may be selected from 1,6-hexanediol diacrylate, polyethyleneglycol diacrylate, polybutadiene diacrylate, tetraethylene glycol diacrylate, 3-methyl-1,5-pentanediol diacrylate, ethoxylated bisphenol A diacrylate, dipropylene glycol diacrylate, ethoxylated hexanediol diacrylate, 1,10-decanediol diacrylate, ester diacrylate, alkoxylated diacrylate, tricyclodecanedimethanol diacrylate, propoxylated neopentyl glycol diacrylate, pentaerythrityl tetraacrylate, trimethylolpropane triacrylate, ethoxylated trimethylolpropane triacrylate, ditrimethylolpropane tetraacrylate, tris(2-hydroxyethyl)isocyanurate triacrylate, di pentaeryth rityl pentaacrylate, pentaerythrityl triacrylate, propoxylated glyceryl triacrylate, aliphatic urethane triacrylate, aliphatic urethane diacrylate, aromatic urethane diacrylate, aromatic urethane triacrylate, aromatic urethane hexaacrylate, polyester hexaacrylate, epoxidized soybean oil diacrylate.

A polyfunctional biomonomer may be used, moreover, in the form of a biobased methacrylate. This may be selected from triethylene glycol dimethacrylate, ethylene glycol dimethacrylate, polyethylene glycol dimethacrylate, 1,4-butanediol dimethacrylate, diethylene glycol dimethacrylate, 1,6-hexanediol dimethacrylate, 1,10-decanediol dimethacrylate, 1,3-butylene glycol dimethacrylate, tricyclodecanedimethanol dimethacrylate, trimethylolpropane trimethacrylate.

It is possible to use fatty acids in the form of plant oils. This may be selected from coconut oil, germ oil, rapeseed oil, cotton seed oil, olive oil, palm oil, peanut oil, safflower oil, sesame oil, soybean oil, sunflower oil, almond oil, beechnut oil, para nut oil, cashew oil, hazelnut oil, macadamia oil, mongongo oil, pecan oil, pistachio oil, walnut oil, pumpkin seed oil, grapefruit kernel oil, lemon oil, orange oil, bitter melon oil, calabash oil, cucurbita oil, butternut seed oil, egusi seed oil, watermelon seed oil, borage seed oil, blackcurrant seed oil, blackseed oil, acai oil, evening primrose oil, linseed oil, amaranth oil, apricot kernel oil, apple seed oil, argan oil, avocado oil, babassu oil, behen oil, sal nut oil, Cape chestnut oil, algoraba oil, cocoa butter, cocklebur oil, cohune oil, coriander seed oil, date seed oil, dika oil, grapeseed oil, hemp oil, kapok seed oil, kenaf seed oil, lallemantia oil, marula oil, mustard oil, ramtil oil, nutmeg butter, ocher seed oil, perilla seed oil, persimmon seed oil, pequi oil, pilli nut oil, pomegranate oil, poppy oil, placaxi oil, plum kernel oil, quinoa oil, rice oil, sacha inchi oil, sapote oil, patawa oil, shea butter, taramira oil, tea seed oil, earth almond oil, tobacco seed oil, tomato seed oil, wheatgerm oil, castor oil, camelina oil, radish oil, Salicornia oil, tung oil, copaiba oleoresin, jatropha oil, jojoba oil, nagkesar oil, pongamia oil, dammar oil, stilingia oil, artichoke oil, murumuru butter, balanos oil, bladderpod oil, macassar kernel oil, burdock root oil, buriti oil, kukui nut oil, carrot seed oil, cuphea oil, mango oil, passionflower oil, rosehip kernel oil, rubber seed oil, sea buckthorn oil, tamanu oil, tonka bean oil.

It is possible, furthermore, to use a fatty acid in the form of an essence oil. This may be selected from oud oil, ajowan oil, angelica root oil, aniseed oil, asafoetida oil, basil oil, peru balsam, laurel oil, bergamot oil, black pepper oil, buchu oil, birch oil, camphor oil, calamondin oil, caraway oil, cardamom oil, cedarwood oil, camellia oil, calmus oil, cinnamon oil, lemon oil, lemongrass oil, sage oil, clove oil, coffee oil, coriander oil, costmary oil, costus root oil, cranberry seed oil, cubeba oil, cumin oil, cypress oil, curryleaf oil, davana oil, dill oil, immortell oil, elemi oil, eucalyptus oil, fennel seed oil, galanga oil, galbanum oil, garlic oil, geranium oil, ginger oil, henna oil, strawflower oil, horseradish oil, jasmine oil, juniper berry oil, lavender oil, balm oil, moringa oil, mugwort oil, myrrh oil, neem oil, oregano oil, nard oil, parsley oil, patchouli oil, perilla oil, peppermint oil, pine kernel oil, rosemary oil, sandalwood oil, sassafras oil, savory oil, schisandra berry oil, mint oil, thyme oil.

It is also possible to use a fatty acid in the form of an animal fat and/or oil. This may be selected from fish oil, bear grass, chicken fat, crocodile fat, crocodile oil, cod liver oil, emu oil, lard, goose fat, duck fat, shark liver oil.

In accordance with the invention, the solution thus prepared, comprising the solvent and the arylamide functionalized with a fatty acid group, is deposited in a rotating powder mixer on the surface of the solid fillers. The (meth)acryloyl groups in the reactive (meth)acrylic monomers undergo esterification to form a polymer matrix, which is immobilized on the surface of the inorganic and/or organic filler particles (see FIG. 2). The figure represents, schematically, the process of filler encapsulation and bonding to the polymer binder.

The surface of inorganic and/or organic filler particles is functional through the presence of functional groups, such as a hydroxyl group on the quartz surface, for example. These functional hydroxyl groups are used as immobilization centers for the biologically derived fatty acid monomer molecules of the invention. The fatty acid-acrylomide molecules chemosorb on the filler surface and form a reactive (meth)acrylate layer with organic chains which point outward from the surface. The water droplet applied to the quartz surface of the quartz particles, modified with the—for example—olive oil-based acrylic monomer, remains for more than 240 seconds on the surface of the filler particles compacted in this way to form a compact. The same effect is achieved when using other oil-based (meth)acrylates. The double bond connected to the (meth)acrylamide is located close to the filler surface. A morphology of this kind enables the construction of a uniform organic layer on the filler surface during the polymerization. Moreover, the double bond of the unsaturated fatty acids, which is at a distance from the filler surface, participates in the process of copolymerization with the matrix monomers. This dimensional specificity in the bonding between matrix and filler increases the impact strength and the thermal cycling stability, reduces the effects of the degree of crosslinking, and so leads to a reduction in the fragility of the molding.

As well as the process, the invention also relates to a hydrophobic and reactive, inorganic and/or organic filler produced by the process of the invention.

Inorganic and organic fillers in accordance with the invention have a surface, preferably treated with at least one biobased (meth)acrylate monomer, which comprises a biologically derived fatty acid group with a group esterified to the (meth)acrylate group. Composite materials which comprise these laid-open, inorganic and/or organic fillers with a surface treated with at least one biobased (meth)acrylate monomer are able to show the reduction in the stresses during the curing of the molding. The cured molding laid open here may have reduced stresses, thereby providing satisfactory improvement in the mechanical properties of the polymerized composite.

The invention additionally relates to the use of such a filler as an adjuvant to a polymer-based casting compound. A casting compound of this kind is used for producing cast and cured moldings, in the form, for example, of kitchen sinks, shower trays, etc.

The invention relates, lastly, to a cured molding in the form, for example, of a kitchen or sanitary article, e.g., a kitchen sink or shower tray, produced using such a casting compound.

The various features of novelty which characterize the invention are pointed out with particularity in the claims annexed to and forming a part of the disclosure. For a better understanding of the invention, its operating advantages, specific objects attained by its use, reference should be had to the drawings and descriptive matter in which there are illustrated and described preferred embodiments of the invention.

### BRIEF DESCRIPTION OF THE DRAWING

FIG. 1 shows the comparative spectra of the untreated quartz flour (bottom) and of the hydrophobized and activated quartz flour.

FIG. 2 shows a schematic representation of the operation of the filler encapsulation and joining to the polymer binder.

### EXAMPLE OF THE INVENTION

Presented below is an experimental example for illustrating the inorganic and/or organic fillers, including the hydrophobized and activated surface, of the invention, the casting compound of the invention, and the shaping of the invention in detail.

### Example

Hydrophobization and Activation of the Inorganic and/or Organic Fillers

Components used:

a) Inorganic and/or Organic Fillers:

Quartz sand (particle size 0.06 to 0.3 mm, manufacturer: Dorfner GmbH), quartz flour (1 to 50 μm, Dorfner GmbH), cristobalite flour (0.1 to 10 μm, Quartzwerke GmbH), olive stone flour (1.0-100 μm, BioPowder Ltd), olive stone particles (600-800 μm, BioPowder Ltd), peach stone particles (300-600 μm, BioPowder Ltd)

b) Biobased Monomers:

Isobornyl methacrylate (IBOMA, Evonik Performance Materials GmbH), polyethylene glycol 200 dimethacrylate (PEG-200-DMA, Arkema)

c) Methacryloyl Monomers Based on Plant Oil:

Monomer based on olive oil (OBM, North Dakota State University), monomer based on soybean oil (SBM, North Dakota State University)

The compositions for producing hydrophobizing and activating agents are produced by dissolving plant oil-based methacryloyl monomers (OBM and/or SBM, North Dakota State University) in the biobased monomers (IBOMA (Evonik Performance Materials GmbH) and/or PEG-200-DMA (Arkema)). The reaction mixture was sonicated at 35° C. for 40 minutes (Bandelin Super RK 1028 H ultrasound bath) until a clear yellowish solution was obtained. For comparison of the hydrophobizing and activating agents, the compositions were produced as summarized in Table 1. The figures are reported in percent by weight.

TABLE 1

| | Specimen 1 | Specimen 2 | Specimen 3 | Specimen 4 | Specimen 5 |
|---|---|---|---|---|---|
| IBOMA | 90 | 85 | 60 | 20 | |
| PEG-200-DMA | | | 31 | 65 | 90 |
| OBM | 10 | 2 | | 15 | 5 |
| SBM | | 13 | 9 | | 5 |

All of the specimens from Table 1 were used as hydrophobizing and activating agents for treating the inorganic and/or organic fillers in the various proportions in accordance with the specific surface area of the filler particles (0.221 m²/g for quartz sand; 1.5 m²/g for quartz flour; 3.5 m²/g for cristobalite flour; 2.6 m²/g—olive stone flour; 0.32 and 0.27 m²/g for olive stone and peach stone particles respectively). The figures are based on the specific surface area per gram of filler.

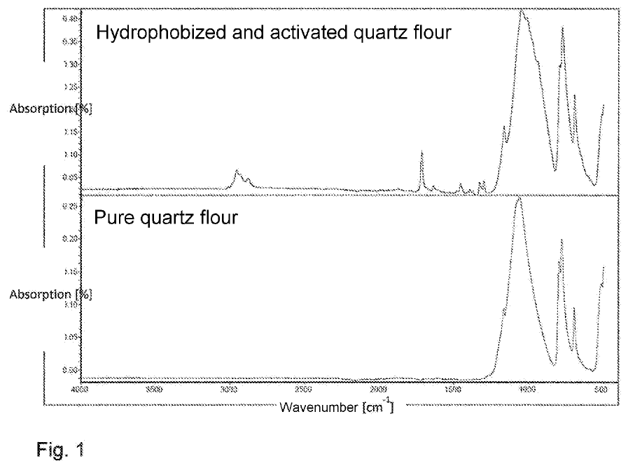

The clear solution of the plant oil-based methacryloyl monomers of specimens 1-5 was used for hydrophobizing and activating the inorganic and/or organic filler surface. The corresponding amount of the solution was added to the fillers, such as quartz sand (particle size 0.06 to 0.3 mm, Dorfner GmbH), quartz flour (1 to 50 μm, Dorfner GmbH), cristobalite flour (0.1 to 10 μm, Quartzwerke GmbH), olive stone flour (1.0-100 μm, BioPowder Ltd), olive stone particles (600-800 μm, BioPowder Ltd), peach stone particles (300-600 μm, BioPowder Ltd) and placed into a mixing cylinder. The cylinder was closed and placed on rotating rolls, in order for the filler particles to be uniformly wetted with the hydrophobizing and activating agent. The mixtures produced in this way were stirred for 2 hours at a rotary speed of 30 rpm. The fillers hydrophobized in this way were subsequently taken from the container and transferred for further use for the production of casting compounds. FIG. 1 shows the IR spectrum of the quartz flour (quartz flour as obtained, bottom) and the IR spectrum of the quartz flour treated with the olive oil-based methacryloyl monomer. An intense peak at around 1650 cm-1 unambiguously confirms the presence of reactive double bonds, which can be copolymerized with the matrix monomers.

Table 2 summarizes the filler compositions hydrophobized and activated with the oil-based monomers of Table 1. The figures are reported in percent by weight.

TABLE 2

| | Specimen 1 | Specimen 2 | Specimen 3 | Specimen 4 | Specimen 5 |
|---|---|---|---|---|---|
| Quartz sand | 90 | 85 | 60 | 40 | 50 |
| 0.06 to 0.3 mm | | | | | |
| Quartz flour | | | 31 | | 20 |
| 1 to 50 μm | | | | | |
| Cristobalite flour | 10 | 2 | | 15 | |
| 0.1 to 10 μm | | | | | |
| Olive stone flour | | 13 | 9 | | 5 |
| 1.0-100 μm | | | | | |
| Olive stone granules | | | | 45 | |
| 600-800 μm | | | | | |
| Peach stone granules | | | | | 25 |
| 300-600 μm | | | | | |

These filler mixtures (specimens 1 to 5) were used for producing the casting compounds and for subsequent curing in the respective mold.

The typical formulation may be described as follows:

23.7 kg of recycled PMMA (XP-95, KFG, Germany) were dissolved in a mixture of 56.3 kg of recycled methyl methacrylate (r-MMA, Monomeros des Valles, Spain), 15 kg of isobornyl methacrylate, Visiomer Terra IBOMA (Evonik Performance Materials, Germany) and 5 kg of biobased ethyl methacrylate (BCH-Bruehl, Germany) until a clear solution was obtained. 0.1 kg of biobased stearic acid (Musim Mas, Singapore) was added to the PMMA solution in monomers. When the stearic acid had dissolved completely, 4.0 kg of Sarbio 6201, polyethylene glycol(200) dimethacrylate (Arkema, France) were added to the PMMA solution. 210 kg of the filler system from specimens 1 to 5 were dispersed in this mixture.

This solution was used for producing specimens 1-5, by dispersing the corresponding hydrophobized filler mixtures, followed by the addition of the initiator system (2 wt %, calculated from the monomer amount) comprising the mixture of Perkadox 16 and Laurox S (Nouryon, Netherland) in a ratio of 1:2.

Following addition of the initiator system and venting for 15 minutes, the casting compound was injected into the closed mold, which for curing was heated at 100° C. for 30 minutes and subsequently cooled, after which the moldings produced were removed from the molds.

In parallel, comparative moldings were produced using a respectively identical casting compound, but containing, rather than the fillers treated in accordance with the invention, the same fillers but untreated, in identical concentration, in order to be able to compare the properties of moldings with inventively treated fillers with the properties of the same moldings with untreated fillers.

The mechanical and thermal properties of the moldings of specimens 1-5 and of the comparative moldings (1 a-5a), produced using the untreated fillers in the same concentrations, were compared with those of the specimens of the invention.

TABLE 3

| | Molding | Molding | Molding | Molding | Molding |
|---|---|---|---|---|---|
| | 1/1a | 2/2a | 3/3a | 4/4a | 5/5a |
| Impact strength, mJ/mm² | 3.7/3.3 | 3.3/3.0 | 3.4/3.2 | 3.4/3.2 | 3.5/3.2 |
| Scratch test | +/+ | +/+ | +/+ | +/+ | +/+ |
| Taber abrasion, mg | 22/20 | 20/20 | 23/21 | 21/19 | 20/19 |
| Resistance to dry heat | +/+ | +/+ | +/+ | +/+ | +/+ |
| Thermal cycling stability | +/+ | +/+ | +/+ | +/+ | +/+ |

For the impact strength measurements, 12 samples with a size of 80×6 mm were cut from the molding. The measurements were carried out using a ZwickRoell HIT P pendulum impact instrument.

For the measurement of the scratch resistance, a sample (100×100 mm) was cut out and tested according to DIN EN 13310 (Erichsen 213 scratch instrument) and the topography before and after scratching was measured (Mitutoyo Surftest SJ 500 P roughness instrument).

For the Taber abrasion test, a sample (100×100 mm) was cut and an abrasion test was carried out with an Elcometer 1720.

The resistance to dry heat is based on the DIN EN 13310 test method, in which the test piece is placed at a temperature of 180° C. for 20 minutes into the center of the molding under test, without leaving visible alterations on the structure of the sink.

The test method of thermal cycling stability is based on the DIN 13310 test method, in which the test molding (kitchen sink) is treated for 1000 cycles with cold-hot water. Hot water (T=90° C.) runs into the sink for 90 seconds, followed by a rest phase of 30 seconds, in which in turn cold water (T=15° C.) runs for 90 seconds. The cycle is ended by 30 seconds of relaxation.

As the measurement results show, virtually all of the moldings of the invention exhibit improved properties relative to the comparative moldings.

Hence there has been a distinct improvement in the impact strength, in some cases by 10% as compared with the comparative molding.

The same is true of the Taber abrasion as well.

All of the moldings of the invention also met the test requirements in relation to scratch resistance, resistance to dry heat, and thermal cycling stability.

FIG. 1 shows the IR spectra of the quartz flour before and after treatment with olive oil-based methacryloyl monomer. Clearly apparent in the spectrum of the treated filler is the double bond of the monomer, which polymerizes with the matrix monomers and forms the less-exposed interface between filler and matrix and also the molding overall. The intense peak of 1650 cm-1 unambiguously confirms the presence of a reactive double bond of the natural oil-based monomer, which is able to copolymerize onto the matrix by grafting.

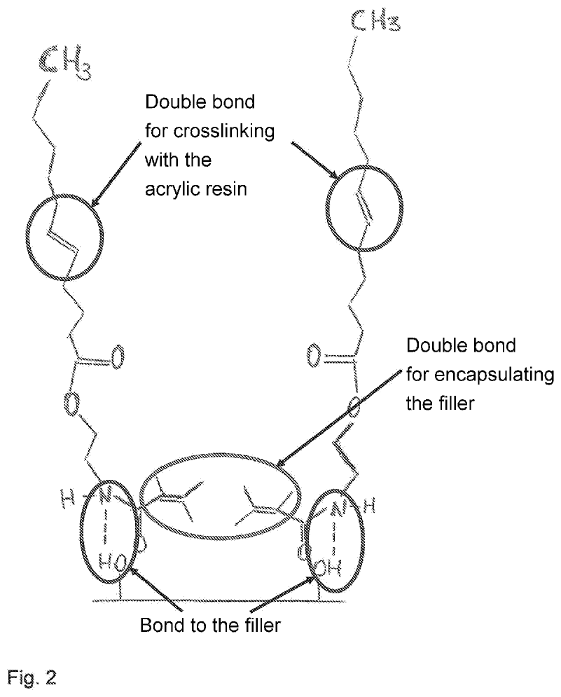

FIG. 2 shows the scheme of the quartz sand surface treated with the olive oil-based methacryloyl polymer, where the double bonds of the methacryloyl part, which form the encapsulation layer on the sand surface, and the double bonds of the labile chains, which copolymerize with the matrix monomers, can be seen. The long CH2—CH2 fatty acid chain enables a flexible response to mechanical and thermal stresses. The amino group of the methacryloyl part of the molecules produces a strong bond to the filler surface.

While specific embodiments of the invention have been shown and described in detail to illustrate the inventive principles, it will be understood that the invention may be embodied otherwise without departing from such principles.

We claim:

1. A process for producing hydrophobic and reactive, inorganic and/or organic fillers, comprising the steps of: (a) providing a filler having a defined surface area, (b) mixing the filler with the solution of at least one hydrophobizing and activating, biologically based reactive compound in a mixing assembly in an amount of 0.15×10⁻² to 5.0×10⁻² g per m² filler surface area at a speed of 20 rpm to 200 rpm for 12 minutes to 120 minutes, (c) evacuating the hydrophobized and activated, inorganic and/or organic filler in a storage bag, a box or a drum, or directly in the casting compound.

2. The process according to claim 1, wherein in step (a) the inorganic filler is selected from SiO₂, Al₂O₃, TiO₂, ZrO₂, Fe₂O₃, ZnO, Cr₂O₅, carbon, metals and metal alloys, SiC, SiN, BN, or a mixture thereof.

3. The process according to claim 1, wherein in step (a) the organic filler is selected from ground fruit stones and/or fruit shells and may be selected from olive stones, peach stones, apricot stones, cherry stones, almond shells, argan shells, walnut shells, or a mixture thereof.

4. The process according to claim 2, wherein in step (a) the inorganic and organic fillers may be used in a combination of both kinds of fillers in any desired mixing ratio.

5. The process according to claim 1, wherein in step (a) the inorganic and organic fillers may have a particle size of 1 μm to 2000 μm.

6. The process according to claim 1, wherein in step (b) the hydrophobizing and activating, biologically based reactive compound is selected from the plant oil-based methacryloyl monomer of the general formula: H₂C═C(R1)C(O)—NH—CH₂—CH₂—C(O)—O—R₂, where R₁ in the case of acryloyl is H and in the case of methacryloyl is CH₃, where R₂ is a fatty acid radical from the plant oil-based oils that reacts in bulk with the N-hydroxyethyl(meth)acrylamide.

7. The process according to claim 1, wherein in step (b) the hydrophobizing and activating, biobased reactive compound is dissolved in the at least one monomer present in the polymer matrix of the molding.

8. The process according to claim 1, wherein the monomer used in step (b) as solvent for the hydrophobizing and activating reactive compounds is selected from monofunctional acrylic monomers such as methyl acrylate, ethyl acrylate, n-butyl acrylate, tert-butyl acrylate, isobutyl acrylate, isodecyl acrylate, dihydroxycyclopentadienyl acrylate, ethyl diglycol acrylate, heptadecyl acrylate, 4-hydroxybutyl acrylate, 2-hydroxyethyl acrylate, hydroxyethylcaprolactone acrylate, polycaprolactone acrylate, hydroxypropyl acrylate, lauryl acrylate, stearyl acrylate, 2-(2-ethoxy)ethyl acrylate, tetrahydroxyfurfuryl acrylate, 2-phenoxyethyl acrylate, ethoxylated 4-phenyl acrylate, trim ethylcyclohexyl acrylate, octyldecyl acrylate, tridecyl acrylate, ethoxylate 4-nonylphenyl acrylate, isobornyl acrylate, cyclic trimethylolpropane formal acrylate, ethoxylated 4-lauryl acrylate, polyester acrylate, hyperbranched polyester acrylate, melamine acrylate, silicone acrylate, epoxy acrylate.

9. The process according to claim 1, wherein the monomer used in step (b) as solvent for the hydrophobizing and activating, biobased reactive compounds is selected from monofunctional methacrylic monomers such as methyl methacrylate, ethyl methacrylate, n-butyl methacrylate, isobutyl methacrylate, tert-butyl methacrylate, behenyl methacrylate, behenylpolyethylene glycol methacrylate, cyclohexyl methacrylate, isodecyl methacrylate, 2-ethylhexyl methacrylate, lauryl methacrylate, stearyl methacrylate, stearylpolyethylene glycol methacrylate, isotridecyl methacrylate, ureidomethacrylate, tetrahydrofurfuryl methacrylate, phenoxyethyl methacrylate, isobornyl methacrylate, methoxypolyethylene glycol methacrylate, glycidyl methacrylate, glycerol formal methacrylate, lauryltetradecyl methacrylate.

10. The process according to claim 1, wherein the monomer used in step (b) as solvent for the hydrophobizing and activating, biobased reactive compounds is selected from polyfunctional acrylic monomers such as 1,6-hexanediol diacrylate, polyethyleneglycol diacrylate, polybutadiene diacrylate, tetraethylene glycol diacrylate, 3-methyl-1,5-pentanediol diacrylate, ethoxylated bisphenol A diacrylate, dipropylene glycol diacrylate, ethoxylated hexanediol diacrylate, 1,10-decanediol diacrylate, ester diacrylate, alkoxylated diacrylate, tricyclodecanedimethanol diacrylate, propoxylated neopentyl glycol diacrylate, pentaerythrityl tetraacrylate, trimethylolpropane triacrylate, ethoxylated trimethylolpropane triacrylate, ditrimethylolpropane tetraacrylate, tris(2-hydroxyethyl)isocyanurate triacrylate, di pentaerythrityl pentaacrylate, pentaerythrityl triacrylate, propoxylated glyceryl triacrylate, aliphatic urethane triacrylate, aliphatic urethane diacrylate, aromatic urethane diacrylate, aromatic urethane triacrylate, aromatic urethane hexaacrylate, polyester hexaacrylate, epoxidized soybean oil diacrylate.

11. The process according to claim 1, wherein the monomer used in step (b) as solvent for the hydrophobizing and activating, biobased reactive compounds is selected from polyfunctional methacrylic monomers such as triethylene glycol dimethacrylate, ethylene glycol dimethacrylate, polyethylene glycol dimethacrylate, 1,4-butanediol dimethacrylate, diethylene glycol dimethacrylate, 1,6-hexanediol dimethacrylate, 1,10-decanediol dimethacrylate, 1,3-butylene glycol dimethacrylate, tricyclodecanedimethanol dimethacrylate, trimethylolpropane trimethacrylate.

12. The process according to claim 6, wherein the oil used is a plant oil.

13. The process according to claim 12, wherein the plant oil is selected from coconut oil, germ oil, rapeseed oil, cotton seed oil, olive oil, palm oil, peanut oil, safflower oil, sesame oil, soybean oil, sunflower oil, almond oil, beechnut oil, para nut oil, cashew oil, hazelnut oil, macadamia oil, mongongo oil, pecan oil, pistachio oil, walnut oil, pumpkin seed oil, grapefruit kernel oil, lemon oil, orange oil, bitter melon oil, calabash oil, cucurbita oil, butternut seed oil, egusi seed oil, watermelon seed oil, borage seed oil, blackcurrant seed oil, blackseed oil, acai oil, evening primrose oil, linseed oil, amaranth oil, apricot kernel oil, apple seed oil, argan oil, avocado oil, babassu oil, behen oil, sal nut oil, Cape chestnut oil, algoraba oil, cocoa butter, cocklebur oil, cohune oil, coriander seed oil, date seed oil, dika oil, grapeseed oil, hemp oil, kapok seed oil, kenaf seed oil, lallemantia oil, marula oil, mustard oil, ramtil oil, nutmeg butter, ocher seed oil, perilla seed oil, persimmon seed oil, pequi oil, pilli nut oil, pomegranate oil, poppy oil, placaxi oil, plum kernel oil, quinoa oil, rice oil, sacha inchi oil, sapote oil, patawa oil, shea butter, taramira oil, tea seed oil, earth almond oil, tobacco seed oil, tomato seed oil, wheatgerm oil, castor oil, camelina oil, radish oil, Salicornia oil, tung oil, copaiba oleoresin, jatropha oil, jojoba oil, nagkesar oil, pongamia oil, dammar oil, stilingia oil, artichoke oil, murumuru butter, balanos oil, bladderpod oil, macassar kernel oil, burdock root oil, buriti oil, kukui nut oil, carrot seed oil, cuphea oil, mango oil, passionflower oil, rosehip kernel oil, rubber seed oil, sea buckthorn oil, tamanu oil, tonka bean oil.

14. The process according to claim 6, wherein the oil used is an essential oil.

15. The process according to claim 14, wherein the essential oil is selected from oud oil, ajowan oil, angelica root oil, aniseed oil, asafoetida oil, basil oil, peru balsam, laurel oil, bergamot oil, black pepper oil, buchu oil, birch oil, camphor oil, calamondin oil, caraway oil, cardamom oil, cedarwood oil, camellia oil, calmus oil, cinnamon oil, lemon oil, lemongrass oil, sage oil, clove oil, coffee oil, coriander oil, costmary oil, costus root oil, cranberry seed oil, cubeba oil, cumin oil, cypress oil, curryleaf oil, davana oil, dill oil, immortell oil, elemi oil, eucalyptus oil, fennel seed oil, galanga oil, galbanum oil, garlic oil, geranium oil, ginger oil, henna oil, strawflower oil, horseradish oil, jasmine oil, juniper berry oil, lavender oil, balm oil, moringa oil, mugwort oil, myrrh oil, neem oil, oregano oil, nard oil, parsley oil, patchouli oil, perilla oil, peppermint oil, pine kernel oil, rosemary oil, sandalwood oil, sassafras oil, savory oil, schisandra berry oil, mint oil, thyme oil.

16. The process according to claim 6, wherein the oil used is selected from animal fat and/or oil. This may be selected from fish oil, bear grass, chicken fat, crocodile fat, crocodile oil, cod liver oil, emu oil, lard, goose fat, duck fat, shark liver oil.

17. The process according to claim 1, wherein in step (b) the concentration of the hydrophobizing and activating, biobased reactive compound in the monomer which is in the polymer matrix of the molding and is used as solvent ought to be from 1 to 20 wt %, preferably from 3 to 17.5 wt %, more particularly from 5 to 15 wt %.

18. A hydrophobic and reactive, inorganic or organic filler, produced by the process according to claim 1.

19. Use of a filler according to claim 18 as adjuvant to a polymer-based casting compound for producing a composite molding or as part of a composite dental filling system.

20. A molding produced from a casting compound according to claim 19.

21. The molding according to claim 20, which is a kitchen sink, a sanitary article in the form of a wash basin, a shower tray, a bath tub, a WC, or a bidet.
